(19) United States
(12) Patent Application Publication
(10) Pub. No.: US 2015/0010972 A1
(43) Pub. Date: Jan. 8, 2015

(54) METHOD FOR PRODUCING LACTIC ACID

(71) Applicant: KAO CORPORATION, Tokyo (JP)

(72) Inventors: Yutaka Irie, Wakayama-shi (JP); Shingo Koyama, Wakayama-shi (JP); Masahiro Noba, Wakayama-shi (JP); Taiki Urakawa, Wakayama-shi (JP); Satoshi Nakahara, Wakayama-shi (JP)

(73) Assignee: KAO CORPORATION, Tokyo (JP)

(21) Appl. No.: 14/384,072

(22) PCT Filed: Apr. 18, 2013
(86) PCT No.: PCT/JP2013/061519

§ 371 Date: Sep. 9, 2014

(30) Foreign Application Priority Data
Apr. 27, 2012 (JP) 2012-102305

(51) Int. Cl. C12P 7/56

(52) U.S. Cl. CPC C12P 7/56
USPC 435/139

(57) ABSTRACT

Provided is a method for producing lactic acid by use of a filamentous fungus, in which the productivity of lactic acid can be maintained at high level even when lactic acid is produced continuously.

The method for producing lactic acid of the present invention includes a first fermentation step of carrying out fermentation by use of one or more fungus cells selected from the group consisting of pellet-form filamentous fungus cells and immobilized filamentous fungus cells in a liquid culture medium having a phosphate ion concentration of less than 0.007% by mass and containing a carbon source, to thereby produce lactic acid.

### FIELD OF THE INVENTION

The present invention relates to a method for producing lactic acid.

### BACKGROUND OF THE INVENTION

Filamentous fungi such as Rhizopus oryzae are likely to form pellets. It has been known that employment of such filamentous fungi for production of lactic acid facilitates separation of the thus-fermented product from a culture medium, and enables a continuous production process (Patent Document 1). For example, it has been reported that lactic acid was continuously produced over 25 days (25 cycles) through a semi-batch reaction process employing Rhizopus oryzae pellets (Non-Patent Document 1).

It has also been reported that when a filamentous fungus is cultured in a culture medium in which the amounts of phosphate ions, potassium ions, sodium ions, magnesium ions and calcium ions, respectively, are controlled from 5 to 60 mM, 5 to 60 mM, 2 to 50 mM, 0.5 to 9 mM and 0.5 to 12 mM, excessive formation of pulps or pellets of the filamentous fungus is suppressed and the pulps and the pellets are maintained in a mixed state, whereby the yield of an unsaturated fatty acid is considerably increased (Patent Document 2).

### CITATION LIST

### Patent Document

Patent Document 1: JP-H06-253871A
Patent Document 2: WO 98/29558

### Non-Patent Document

Non-Patent Document 1: Journal of Industrial Microbiology and Biotechnology, Vol. 38 (2011) 565-571

### SUMMARY OF THE INVENTION

The present invention provides a method for producing lactic acid, the method comprising a first fermentation step of carrying out fermentation by use of one or more fungus cells selected from the group consisting of pellet-form filamentous fungus cells and immobilized filamentous fungus cells in a liquid culture medium having a phosphate ion concentration of less than 0.007% by mass and containing a carbon source, to thereby produce lactic acid.

### DETAILED DESCRIPTION OF THE INVENTION

The present inventors found that when lactic acid is continuously produced through fermentation by use of a pellet-form filamentous fungus or an immobilized filamentous fungus in a liquid culture medium containing a carbon source, the amount of alcohol produced increases over time, and the productivity of lactic acid is reduced.

An object of the present invention is to provide a method for producing lactic acid by use of a filamentous fungus, in which the productivity of lactic acid can be maintained at high level even when lactic acid is produced continuously.

The present inventors have conducted extensive studies for investigating the cause of the aforementioned reduction in productivity of lactic acid, and as a result have found that when the concentration of a particular component is adjusted to be less than a specific level in a liquid culture medium containing a carbon source, a pellet-form filamentous fungus or an immobilized filamentous fungus is maintained in a mycelial form, and the productivity of lactic acid can be maintained at high level even when lactic acid is produced continuously.

According to the present invention, there is provided a method for producing lactic acid, in which the productivity of lactic acid can be maintained at high level even when lactic acid is produced continuously, while a pellet-form filamentous fungus or an immobilized filamentous fungus is maintained in a mycelial form.

The method for producing lactic acid of the present invention comprises a first fermentation step of carrying out fermentation by use of one or more fungus cells selected from the group consisting of pellet-form filamentous fungus cells and immobilized filamentous fungus cells (hereinafter the filamentous fungus cells may be referred to simply as “cells”) in a liquid culture medium having a phosphate ion concentration of less than 0.007% by mass and containing a carbon source, to thereby produce lactic acid.

Examples of the filamentous fungus employed in the present invention include microorganisms belonging to the genus Rhizopus, the genus Aspergillus, and the genus Mucor. Of these, microorganisms belonging to the genus Rhizopus are preferred. Specifically, preferred are Rhizopus oryzae, Aspergillus oryzae, Aspergillus niger, Aspergillus terreus, and Mucor mandshuricus, and Rhizopus oryzae being more preferred.

The filamentous fungus employed in the present invention may be a single form of pellet-form filamentous fungus or immobilized filamentous fungus, or may be a mixture of pellet-form filamentous fungus and immobilized filamentous fungus. As used herein, the term “pellet-form filamentous fungus” refers to pellets of a filamentous fungus having mycelia spontaneously formed through liquid culture, and having a size of several hundreds of μm to several mm. As used herein, the term “immobilized filamentous fungus” refers to a filamentous fungus supported on or embedded in a carrier.

The pellet-form filamentous fungus or immobilized filamentous fungus employed may be a commercially available one, or may be prepared through the following step.

### (Step of Preparing Pellet-Form Filamentous Fungus)

The pellet-form filamentous fungus may be prepared through culturing.

The culture medium employed may be any of a synthetic medium, a natural medium, and a semi-synthetic medium supplemented with a natural component, so long as it is a liquid culture medium capable of growing a filamentous fungus. The culture medium generally contains, for example, a carbon source, a nitrogen source, and an inorganic salt, and the composition of these components may be appropriately selectively determined. The phosphate ion concentration of the culture medium is not necessarily less than 0.007% by mass, and may be appropriately adjusted to be a level generally employed for culturing a filamentous fungus.

The culture temperature is preferably from 20 to 40° C., more preferably from 25 to 30° C. The initial pH (25° C.) of the culture medium is preferably from 3 to 7, more preferably from 4 to 6.

Culturing may be carried out through any known method. For example, after inoculation of filamentous fungal spores into a liquid culture medium, mycelia are germinated from the spores, and cells are produced from the mycelia, followed by pelletization. This culturing is generally carried out under aerobic conditions. Aeration is carried out preferably at from 0.25 to 4 vvm, more preferably at from 0.5 to 2 vvm. The culture period is preferably from 30 minutes to seven days, more preferably from 0.5 to six days, much more preferably from one to five days, after inoculation of the filamentous fungal spores into the liquid culture medium. The culture vessel employed for culturing may be appropriately selected from among conventionally known ones. Specific examples of the culture vessel include an aeration stirring culture vessel, a bubble column culture vessel, and a fluidized bed culture vessel.

After completion of culturing, the pellet-form filamentous fungus may be removed from the culture vessel together with the culture medium, and may be employed in the subsequent step after separation/recovery through a simple operation such as filtration or centrifugation.

Alternatively, the subsequent step may be carried out in the same culture vessel while leaving the pellet-form filamentous fungus in the culture vessel.

This step may be further divided into two or more sub-steps.

### (Step of Preparing Immobilized Filamentous Fungus)

The immobilized filamentous fungus may be prepared through culturing.

Culturing may be carried out through any known method. For example, after inoculation of filamentous fungal spores into a liquid culture medium containing a filamentous-fungus-immobilizing carrier, mycelia are germinated from the spores, and immobilized filamentous fungus is prepared from the mycelia trapped in the carrier. Examples of the material of the filamentous-fungus-immobilizing carrier include a urethane polymer, an olefin polymer, a diene polymer, a condensation polymer, a silicone polymer, and a fluoropolymer. The filamentous-fungus-immobilizing carrier may assume any shape; for example, a shape of flat plate, multilayer plate, wavy plate, tetrahedron, sphere, cord, net, circular column, lattice, or circular cylinder. The filamentous-fungus-immobilizing carrier is preferably in the form of, for example, foam, flake, sheet, hollow product, or resin molded product, more preferably in the form of foam. The size of the filamentous-fungus-immobilizing carrier is preferably from 0.1 mm to 10 mm, more preferably from 0.5 to 5 mm, much more preferably from 0.7 to 2 mm.

The culture medium and culture vessel employed for immobilization of the filamentous fungus may be the same as those for the aforementioned pellet-form filamentous fungus. Also, the culture conditions for immobilization of the filamentous fungus may be the same as those for the aforementioned pellet-form filamentous fungus. After completion of culturing, the immobilized filamentous fungus may be separated and recovered in a manner similar to the case of the pellet-form filamentous fungus, and may be employed in the subsequent step. Alternatively, the subsequent step may be carried out in the same culture vessel while leaving the immobilized filamentous fungus in the culture vessel.

This step may be further divided into two or more sub-steps.

### <First Fermentation Step>

In this step, a carbon source is fermented by use of cells, to thereby produce lactic acid. The lactic acid may be any of an L-form, an R-form, and a racemic mixture.

No particular limitation is imposed on the culture medium employed in this step, so long as it is a liquid culture medium containing a carbon source, and the phosphate ion concentration of the culture medium is adjusted to be less than a specific level. The culture medium may contain, for example, a nitrogen source, an inorganic salt other than a phosphate salt, or a vitamin. When the carbon source contains any of the aforementioned nutrient sources at a concentration suitable for culturing, only the carbon source may be employed.

The phosphate ion concentration of the culture medium employed in this step is less than 0.007% by mass. In order to maintain the pellet-form filamentous fungus in a mycelial form, and to maintain the productivity of lactic acid at high level, the phosphate ion concentration of the culture medium is preferably 0.006% by mass or less, more preferably 0.005% by mass or less, still more preferably 0.004% by mass or less, much more preferably 0.003% by mass or less. Meanwhile, no particular limitation is imposed on the lower limit of the phosphate ion concentration of the culture medium, and the phosphate ion concentration may be 0% by mass (i.e., the culture medium may contain no phosphate ions). As used herein, the expression “phosphate ion concentration of 0% by mass” encompasses the case where the phosphate ion concentration of a culture medium as measured through enzymatic colorimetry is equal to or less than the detection limit. The phosphate ion concentration is from 0 to less than 0.007% by mass, preferably from 0 to 0.006% by mass, more preferably from 0 to 0.005% by mass, still more preferably from 0 to 0.004% by mass, much more preferably from 0 to 0.003% by mass. The reason why such a range is preferred has not yet been elucidated. However, the present inventors have considered that when the phosphate ion concentration falls within such a range, excessive proliferation of the filamentous fungus is suppressed, and the filamentous fungus is maintained in a mycelial form. When the culture medium employed in this step contains phosphate ions, the ions may be contained in the form of phosphate salt. Specific examples of the phosphate salt include the same as those exemplified in the below-described second fermentation step.

The culture medium employed in this step contains a carbon source, and examples of the carbon source include saccharides. Specific examples of the saccharides include glucose, fructose, xylose, and sucrose. These saccharides may be employed singly or in combination of two or more species. Particularly, glucose or fructose is preferably employed, from the viewpoint of maintenance of high productivity of lactic acid.

The carbon source employed in this step may be a sugar solution containing such a saccharide. Specific examples of the sugar solution include a sugar solution derived from starch, syrup (molasses), and a sugar solution derived from lignocellulosic biomass. These sugar solutions may be employed singly or in combination of two or more species. As used herein, the term “lignocellulosic biomass” refers to biomass containing cellulose, hemicellulose, or lignin as a main component. Specific examples of the lignocellulosic biomass include rice straw, chaff, wheat straw, bagasse, palm shell, corn cob, weed, wood, and pulp or paper produced therefrom. Examples of the starch include extracts of cereals such as corn, and extracts of beans such as soybean. Examples of the syrup include syrups derived from sugar cane and sugar beet.

In order to maintain the productivity of lactic acid at high level, the initial carbon concentration of the culture medium is preferably 1% by mass or more, more preferably 3% by mass or more, much more preferably 5% by mass or more, and is preferably 40% by mass or less, more preferably 30% by mass or less, much more preferably 20% by mass or less. Thus, the initial carbon concentration of the culture medium is preferably from 1 to 40% by mass, more preferably from 3 to 30% by mass, much more preferably from 5 to 20% by mass.

The culture medium employed in this step may contain a nitrogen source. Specific examples of the nitrogen source include nitrogen-containing compounds such as urea, ammonium nitrate, potassium nitrate, and sodium nitrate. In order to maintain the productivity of lactic acid at high level, the initial nitrogen concentration of the culture medium is preferably from 0.01 to 1% by mass, more preferably from 0.02 to 0.8% by mass, much more preferably from 0.04 to 0.6% by mass.

The culture medium employed in this step may contain a sulfate salt. Specific examples of the sulfate salt include magnesium sulfate, zinc sulfate, potassium sulfate, and sodium sulfate. In order to maintain the productivity of lactic acid at high level, the initial sulfate ion concentration of the culture medium is preferably from 0.001 to 0.1% by mass, more preferably from 0.005 to 0.08% by mass, much more preferably from 0.01 to 0.04% by mass.

The culture medium employed in this step may contain a magnesium salt. Specific examples of the magnesium salt include magnesium sulfate, magnesium nitrate, and magnesium chloride. In order to maintain the productivity of lactic acid at high level, the initial magnesium ion concentration of the culture medium is preferably from 0 to 0.5% by mass, more preferably from 0.001 to 0.2% by mass, much more preferably from 0.002 to 0.1% by mass.

The culture medium employed in this step may contain a zinc salt. Specific examples of the zinc salt include zinc sulfate, zinc nitrate, and zinc chloride. In order to maintain the productivity of lactic acid at high level, the initial zinc ion concentration of the culture medium is preferably from 0 to 0.1% by mass, more preferably from 0.00001 to 0.01% by mass, much more preferably from 0.00005 to 0.005% by mass.

The culture temperature is preferably from 20 to 40° C., more preferably from 30 to 37° C. The pH (25° C.) of the culture medium is preferably from 2 to 7, more preferably from 4 to 6, from the viewpoints of growth of cells, as well as the productivity of lactic acid. Adjustment of pH may be carried out by use of a base such as calcium hydroxide, sodium hydroxide, calcium carbonate, or ammonia, or an acid such as sulfuric acid or hydrochloric acid.

Conditions for culturing may be appropriately determined; i.e., culturing may be carried out under anaerobic conditions or aerobic conditions. Under aerobic conditions, aeration is carried out preferably at from 0.25 to 4 vvm, more preferably at from 0.5 to 2 vvm. The culture vessel employed for culturing may be appropriately selected from among conventionally known ones. In order to improve the production rate of lactic acid, the culture vessel employed is preferably an aeration stirring culture vessel, a bubble column culture vessel, or a fluidized bed culture vessel.

This step may be carried out by, for example, inoculating cells into any of the aforementioned culture media. Alternatively, this step may be carried out by adding any of the aforementioned culture media into the above-prepared cells remaining in the culture vessel.

This step may be carried out through any of a batch process, a semi-batch process, and a continuous process. Preferably, a continuous process is employed from the viewpoint of improvement of productivity.

For example, when this step is carried out through a semi-batch process, the cells may be separated from the resultant fermentation mixture, and fermentation may be further carried out by adding a culture medium to the thus-separated/recovered cells. When this step is carried out through a continuous process, a specific amount of a culture medium may be supplied to the fermentation vessel at a specific rate while the same amount of the resultant fermentation mixture is removed therefrom. In such a case, the liquid level in the fermentation vessel may be controlled by means of, for example, a liquid level sensor so that the liquid level is maintained constant. Meanwhile, only a carbon source may be supplied during fermentation. In such a case, supply of the carbon source may be controlled on the basis of flow rate or glucose concentration.

### <Second Fermentation Step>

In the present invention, the first fermentation step may be followed by the second fermentation step, for the purposes of activating mycelia, and maintaining the productivity of lactic acid at high level. Specifically, the first fermentation step is completed at the time when the percent maintenance of lactic acid production rate becomes 50 to 95% in the first fermentation step, and, in the second fermentation step, fermentation is carried out by use of the cells employed in the first fermentation step in a liquid culture medium having a phosphate ion concentration of from 0.007% by mass to 1% by mass and containing a carbon source.

This step can restore the productivity of lactic acid, which has been lowered through long-term fermentation/production. The mechanism by which the productivity of lactic acid is restored in this step has not yet been elucidated. However, conceivably, mycelia whose activity has been lowered due to phosphorus depletion are reactivated through supply of an appropriate amount of phosphate.

In order to activate mycelia, and to maintain the productivity of lactic acid at high level, this step is carried out at the time when the percent maintenance of lactic acid production rate in the first fermentation step becomes preferably 50% or more, more preferably 60% or more, much more preferably 70% or more, and becomes preferably 95% or less, more preferably 90% or less, much more preferably 85% or less. The percent maintenance of lactic acid production rate is generally from 50 to 95%, preferably from 50 to 90%, more preferably from 60 to 90%, still more preferably from 70 to 90%, much more preferably from 70 to 85%.

As used herein, the “percent maintenance of lactic acid production rate” is determined by use of the following formula (i).

T [%]=Vt [g/L/h]/Vi [g/L/h]×100 (i)

[In formula (i), T represents the percent maintenance of lactic acid production rate [%]; Vt represents lactic acid production rate in a sample [g/L/h]; and Vi represents the management value of lactic acid production rate [g/L/h].]

In formula (i), lactic acid production rate [g/L/h] is determined by dividing the lactic acid concentration of a sample (g/L) by fermentation time (h). The management value of lactic acid production rate (Vi) is determined on the basis of the relationship between fermentation time and the lactic acid concentration of a fermentation mixture in the first fermentation step. The management value of lactic acid production rate may be determined on the basis of the relationship between fermentation time and the lactic acid concentration of a fermentation mixture, which the relationship has been obtained prior to actual operation. Alternatively, the management value of lactic acid production rate may be determined on the basis of the relationship between fermentation time and the lactic acid concentration of a fermentation mixture, which the relationship is obtained during actual operation. In this case, there may be employed the theoretical value of the concentration of lactic acid (g/L) produced from a carbon source in a liquid culture medium.

The management value of lactic acid production rate (Vi), which may vary with production scale or another factor, is, for example, preferably 0.1 g/L/h or more, more preferably 0.3 g/L/h or more, much more preferably 0.5 g/L/h or more, and is preferably 40 g/L/h or less, more preferably 30 g/L/h or less, much more preferably 20 g/L/h or less. The management value of lactic acid production rate (Vi) is preferably from 0.1 to 40 g/L/h, more preferably from 0.3 to 30 g/L/h, much more preferably from 0.5 to 20 g/L/h.

After completion of the first fermentation step, this step may be carried out by separating the cells from the resultant fermentation mixture, and inoculating the thus-recovered cells into a liquid culture medium newly prepared in this step. Alternatively, after completion of the first fermentation step, this step may be carried out by adding phosphate ions into the liquid culture medium employed in the first step so as to adjust the phosphate ion concentration of the culture medium to a specific level.

The liquid culture medium employed in this step is the same as that employed in the first fermentation step, except that the phosphate ion concentration is from 0.007% by mass to 0.1% by mass. The liquid culture medium may contain, for example, a nitrogen source, an inorganic salt other than a phosphate salt, or a vitamin, so long as it contains a carbon source. When the carbon source is contained with any of the aforementioned nutrient sources at a concentration suitable for culturing, only the carbon source may be employed.

The phosphate ion concentration of the culture medium employed in this step is preferably 0.007% by mass or more, more preferably 0.01% by mass or more, much more preferably 0.03% by mass or more, for the purposes of activating mycelia and maintaining the productivity of lactic acid at high level. The phosphate ion concentration of the culture medium employed in this step is preferably 0.1% by mass or less, more preferably 0.09% by mass or less, much more preferably 0.08% by mass or less for the purpose of maintaining the form of cells. The phosphate ion concentration of the culture medium is preferably from 0.007 to 0.1% by mass, more preferably 0.01 to 0.09% by mass, much more preferably from 0.03 to 0.08% by mass.

The phosphate ions contained in the culture medium employed in this step may be in the form of a phosphate salt. Specific examples of the phosphate salt include dipotassium monohydrogen phosphate, monopotassium dihydrogen phosphate, disodium monohydrogen phosphate, and monosodium dihydrogen phosphate.

In this step, the culture temperature is preferably from 20 to 40° C., more preferably from 30 to 37° C. The pH (25° C.) of the culture medium is preferably from 2 to 7, more preferably from 4 to 6, from the viewpoints of growth of cells, as well as the productivity of lactic acid. Adjustment of pH may be carried out by use of a base such as calcium hydroxide, sodium hydroxide, calcium carbonate, or ammonia, or an acid such as sulfuric acid or hydrochloric acid.

Conditions for culturing may be appropriately determined; i.e., culturing may be carried out under anaerobic conditions or aerobic conditions. Under aerobic conditions, aeration is carried out preferably at from 0.25 to 4 vvm, more preferably at from 0.5 to 2 vvm.

This step is initiated at the time when the following conditions are satisfied; i.e., the temperature of the culture medium is from 20° C. or more to 40° C. or less, and the pH (25° C.) of the culture medium is from 2 to 7. In the case where this step is carried out under aerobic conditions, this step is initiated at the time when the aforementioned conditions are satisfied, and also an aeration condition of from 0.25 vvm or more to 4 vvm or less is satisfied.

In order to activate mycelia, and to maintain the productivity of lactic acid at high level, this step is continued preferably for one hour or longer, more preferably for 12 hours or longer, much more preferably for 24 hours or longer, after initiation of this step. In order to maintain the form of immobilized filamentous fungus, this step is completed preferably within 240 hours, more preferably within 120 hours, still more preferably within 60 hours, much more preferably within 48 hours, after initiation of this step. The fermentation time in this step is preferably from 1 to 240 hours, more preferably from 12 to 120 hours, still more preferably from 24 to 60 hours, much more preferably from 24 to 48 hours.

### <Third Fermentation Step>

In the present invention, when the second fermentation step is carried out, preferably, the third fermentation step is carried out after completion of the second fermentation step, for the purpose of maintaining the productivity of lactic acid at higher level. Specifically, in the third fermentation step, fermentation is carried out by use of the cells employed in the second fermentation step in a liquid culture medium having a phosphate ion concentration of less than 0.007% by mass and containing a carbon source.

The cells employed in the second fermentation step may be separated and recovered from the resultant fermentation mixture after completion of the second fermentation step. The thus-recovered cells may be inoculated into a liquid culture medium newly prepared in this step.

The liquid culture medium employed in this step is the same as that employed in the first fermentation step, and the specific composition of the culture medium is as described above in the first fermentation step.

In the case where the productivity of lactic acid is lowered in the third fermentation step, when the second fermentation step is carried out again, the productivity of lactic acid can be restored.

### <Step of Separation of Cells and Fermentation Mixture>

After completion of the fermentation step, solid-liquid separation may be carried out by means of a filter in the fermentation vessel for separation of the cells from the fermentation mixture. Alternatively, the fermentation mixture may be temporarily removed from the fermentation vessel and subjected to solid-liquid separation by means of, for example, a liquid cyclone or filtration, and then only the cells may be returned to the fermentation vessel.

<Step of Recovering Lactic Acid from Fermentation Mixture after Separation Step>

Lactic acid may be separated and recovered from the fermentation mixture through, for example, a process in which the fermentation mixture obtained in the separation step is concentrated, and then lactic acid is precipitated as an alkaline earth metal salt through crystallization, ion exchange, or solvent extraction, followed by acid degradation of the precipitate; or a process in which lactic acid is distilled and purified as a lactic acid ester, and the ester is hydrolyzed.

In relation to the aforementioned embodiments, the present invention further discloses the following lactic acid production methods.

<1>

A method for producing lactic acid, the method comprising a first fermentation step of carrying out fermentation by use of one or more fungus cells selected from the group consisting of pellet-form filamentous fungus cells and immobilized filamentous fungus cells in a liquid culture medium having a phosphate ion concentration of less than 0.007% by mass and containing a carbon source, to thereby produce lactic acid.

<2>

The method for producing lactic acid according to <1> above, which comprises a second fermentation step of carrying out fermentation by use of the cells employed in the first fermentation step in a liquid culture medium having a phosphate ion concentration of from 0.007% by mass or more to 1% by mass or less and containing a carbon source, the second fermentation step being carried out at the time when the percent maintenance of lactic acid production rate becomes preferably from 50 to 95% in the first fermentation step.

<3>

The method for producing lactic acid according to <2> above, wherein the percent maintenance of lactic acid production rate is preferably 50% or more, more preferably 60% or more, much more preferably 70% or more, and is preferably 90% or less, more preferably 85% or less.

<4>

The method for producing lactic acid according to <2> or <3> above, wherein the percent maintenance of lactic acid production rate is preferably from 50 to 90%, more preferably from 60 to 90%, still more preferably from 70 to 90%, much more preferably from 70 to 85%.

<5>

The method for producing lactic acid according to any one of <2> to <4> above, wherein the percent maintenance of lactic acid production rate is preferably determined by use of the following formula (i):

T [%]=Vt [g/L/h]/Vi [g/L/h]×100 (i)

[wherein T represents the percent maintenance of lactic acid production rate [5]; Vt represents lactic acid production rate in a sample [g/L/h]; and Vi represents the management value of lactic acid production rate [g/L/h]].
<6>

The method for producing lactic acid according to <5> above, wherein the management value of lactic acid production rate is preferably 0.1 g/L/h or more, more preferably 0.3 g/L/h or more, much more preferably 0.5 g/L/h or more, and is preferably 40 g/L/h or less, more preferably 30 g/L/h or less, much more preferably 20 g/L/h or less.

<7>

The method for producing lactic acid according to <5> or <6> above, wherein the management value of lactic acid production rate is preferably from 0.1 to 40 g/L/h, more preferably from 0.3 to 30 g/L/h, much more preferably from 0.5 to 20 g/L/h.

<8>

The method for producing lactic acid according to any one of <2> to <7> above, wherein the second fermentation step is continued preferably for one hour or longer, more preferably for 12 hours or longer, much more preferably for 24 hours or longer, and is completed preferably within 240 hours, more preferably within 120 hours, still more preferably within 60 hours, much more preferably within 48 hours.

<9>

The method for producing lactic acid according to any one of <2> to <8> above, wherein the second fermentation step is carried out preferably for from 1 to 240 hours, more preferably for from 12 to 120 hours, still more preferably for from 24 to 60 hours, much more preferably for from 24 to 48 hours.

<10>

The method for producing lactic acid according to any one of <2> to <9> above, which preferably comprises a third fermentation step of carrying out fermentation by use of the cells employed in the second fermentation step in a liquid culture medium having a phosphate ion concentration of less than 0.007% by mass and containing a carbon source, the third fermentation step being carried out after completion of the second fermentation step.

<11>

The method for producing lactic acid according to any one of <1> to <10> above, which preferably comprises a step of preparing one or more fungus cells selected from the group consisting of pellet-form filamentous fungus cells and immobilized filamentous fungus cells, the step being carried out before the first fermentation step.

<12>

The method for producing lactic acid according to any one of <1> to <11> above, wherein the filamentous fungus preferably belongs to the genus Rhizopus.

<13>

The method for producing lactic acid according to any one of <1> to <12> above, wherein the filamentous fungus is preferably Rhizopus oryzae.

<14>

The method for producing lactic acid according to any one of <1> to <13> above, wherein the carbon source is preferably a saccharide.

<15>

The method for producing lactic acid according to <14> above, wherein the saccharide is preferably one or more species selected from the group consisting of a sugar solution derived from starch, syrup, and a sugar solution derived from lignocellulosic biomass.

<16>

The method for producing lactic acid according to any one of <1> to <15> above, wherein the phosphate ion concentration of the liquid culture medium employed in the first fermentation step is preferably 0.006% by mass or less, more preferably 0.005% by mass or less, still more preferably 0.004% by mass or less, much more preferably 0.003% by mass or less, much more preferably 0% by mass.

<17>

The method for producing lactic acid according to any one of <1> to <15> above, wherein the phosphate ion concentration of the liquid culture medium employed in the first fermentation step is preferably from 0 to less than 0.007% by mass, more preferably from 0 to 0.006% by mass, still more preferably from 0 to 0.005% by mass, much more preferably from 0 to 0.004% by mass, much more preferably from 0 to 0.003% by mass.

<18>

The method for producing lactic acid according to any one of <1> to <15> above, wherein the liquid culture medium employed in the first fermentation step contains no phosphate ions (phosphate ion concentration: 0% by mass), or, when the liquid culture medium contains phosphate ions, the phosphate ion concentration of the liquid culture medium is preferably from 0.006% by mass or less, more preferably 0.005% by mass or less, still more preferably 0.004% by mass or less, much more preferably 0.003% by mass or less.

<19>

The method for producing lactic acid according to any one of <2> to <18> above, wherein the phosphate ion concentration of the liquid culture medium employed in the second fermentation step is preferably from 0.1% by mass or less, more preferably 0.09% by mass or less, much more preferably 0.08% by mass or less, and is preferably 0.007% by mass or more, more preferably 0.01% by mass or more, much more preferably 0.03% by mass or more.

<20>

The method for producing lactic acid according to any one of <2> to <19> above, wherein the phosphate ion concentration of the liquid culture medium employed in the second fermentation step is preferably from 0.007 to 0.1% by mass, more preferably from 0.01 to 0.09% by mass, much more preferably from 0.03 to 0.08% by mass.

<21>

The method for producing lactic acid according to any one of <10> to <20> above, wherein the phosphate ion concentration of the liquid culture medium employed in the third fermentation step is preferably from 0.006% by mass or less, more preferably 0.005% by mass or less, still more preferably 0.004% by mass or less, much more preferably 0.003% by mass or less, much more preferably 0% by mass.

<22>

The method for producing lactic acid according to any one of <10> to <20> above, wherein the phosphate ion concentration of the liquid culture medium employed in the third fermentation step is preferably from 0 to less than 0.007% by mass, more preferably from 0 to 0.006% by mass, still more preferably from 0 to 0.005% by mass, much more preferably from 0 to 0.004% by mass, much more preferably from 0 to 0.003% by mass.

<23>

The method for producing lactic acid according to any one of <10> to <20> above, wherein the liquid culture medium employed in the third fermentation step contains no phosphate ions (phosphate ion concentration: 0% by mass), or, when the liquid culture medium contains phosphate ions, the phosphate ion concentration of the liquid culture medium is preferably from 0.006% by mass or less, more preferably 0.005% by mass or less, still more preferably 0.004% by mass or less, much more preferably 0.003% by mass or less.

<24>

The method for producing lactic acid according to any one of <1> to <23> above, wherein, in one or more steps of the first, second, and third fermentation steps, the initial carbon concentration of the liquid culture medium is preferably 1% by mass or more, more preferably 3% by mass or more, much more preferably 5 mass % or more, and is preferably 40% by mass or less, more preferably 30% by mass or less, much more preferably 20% by mass or less.

<25>

The method for producing lactic acid according to any one of <1> to <24> above, wherein, in one or more steps of the first, second, and third fermentation steps, the initial carbon concentration of the liquid culture medium is preferably from 1 to 40% by mass, more preferably 3 to 30% by mass, much more preferably 5 to 20% by mass.

<26>

The method for producing lactic acid according to any one of <1> to <25> above, wherein, in one or more steps of the first, second, and third fermentation steps, the initial nitrogen concentration of the liquid culture medium is preferably from 0.01 to 1% by mass, more preferably from 0.02 to 0.8% by mass, much more preferably from 0.04 to 0.6% by mass.

<27>

The method for producing lactic acid according to any one of <1> to <26> above, wherein, in one or more steps of the first, second, and third fermentation steps, the initial sulfate ion concentration of the liquid culture medium is preferably from 0.001 to 0.1° by mass, more preferably from 0.005 to 0.08% by mass, much more preferably from 0.01 to 0.04% by mass.

<28>

The method for producing lactic acid according to any one of <1> to <27> above, wherein, in one or more steps of the first, second, and third fermentation steps, the initial magnesium ion concentration of the liquid culture medium is preferably from 0 to 0.5% by mass, more preferably from 0.001 to 0.2% by mass, much more preferably from 0.002 to 0.1% by mass.

<29>

The method for producing lactic acid according to any one of <1> to <28> above, wherein, in one or more steps of the first, second, and third fermentation steps, the initial zinc ion concentration of the liquid culture medium is preferably from 0 to 0.1% by mass, more preferably from 0.00001 to 0.01% by mass, much more preferably from 0.00005 to 0.005% by mass.

<30>

The method for producing lactic acid according to any one of <1> to <29> above, wherein one or more steps of the first, second, and third fermentation steps are preferably carried out through a continuous process in which a specific amount of a liquid culture medium is supplied to a fermentation vessel at a specific rate while the same amount of the resultant fermentation mixture is removed from the fermentation vessel.

<31>

A method for reactivating cells employed for lactic acid production, the method comprising a first fermentation step of carrying out fermentation by use of one or more fungus cells selected from the group consisting of pellet-form filamentous fungus cells and immobilized filamentous fungus cells in a liquid culture medium having a phosphate ion concentration of less than 0.007% by mass and containing a carbon source, to thereby produce lactic acid; and a second fermentation step of carrying out fermentation by use of the cells employed in the first fermentation step in a liquid culture medium having a phosphate ion concentration of from 0.007% by mass or more to 1% by mass or less and containing a carbon source, the second fermentation step being carried out at the time when the percent maintenance of lactic acid production rate becomes preferably from 50 to 95% in the first fermentation step.

### EXAMPLES

### Analysis Method

### Measurement of Components Through High-Performance Liquid Chromatography (HPLC)

A fermentation mixture was appropriately diluted with 0.0085N aqueous sulfuric acid solution, and then subjected to filtration by means of a cellulose acetate-made membrane filter having a pore size of 0.22 μm (product of ADVANTEC), to thereby prepare a sample for HPLC analysis. HPLC analysis conditions are as follows.
Column: ICSep ICE-ION-300
Eluent: 0.0085N sulfuric acid (0.4 mL/min)
Detection method: RI (HITACHI, L-2490)
Column temperature: 40° C.
Injection amount: 20 μL
Retention time: 40 min

The retention times of the respective components in this analysis system are as follows.
Glucose: 16 min
Lactic acid: 23 min
Ethanol: 34 min

### Culture Example 1

### Preparation of Pellet-Form Filamentous Fungus

### Preparation of Spore Suspension

There was employed filamentous fungus R. oryzae NBRC5384 strain obtained from National Institute of Technology and Evaluation (NITE). Cells of the filamentous fungus strain were streaked and applied onto a slat agar medium (Difco Potato Dextrose Agar, Becton, Dickinson and Company) formed in a test tube, and static culture was carried out at 25° C., followed by periodic passage culture.

Sterile distilled water (10 mL) was added to the test tube containing proliferated cells, and then the test tube was stirred by means of a touch mixer for four minutes, to thereby recover spores. The thus-recovered spores were diluted with sterile distilled water, to thereby prepare a spore suspension (1×10⁶ spores/mL).

### Pelletization of Filamentous Fungus

A pellet-form filamentous fungus was prepared through the following two-stage culturing.

In the first stage, a 200-mL baffled Erlenmeyer flask containing PDB medium (Difco Potato Dextrose Broth, Becton, Dickinson and Company) (60 mL) was sterilized; the above-prepared spore suspension was inoculated into the PDB medium (1×10⁴ spores/mL); and culturing was carried out at 27° C. and 100 r/m (PRXYg-98R, product of PRECI) for three days.

In the second stage, 2-L airlift-type fermentation vessel containing 2 L of pelletization medium (10% by mass of glucose (product of Wako Pure Chemical Industries, Ltd.), 0.025% by mass of magnesium sulfate heptahydrate, 0.009% by mass of zinc sulfate heptahydrate, 0.1% by mass of ammonium sulfate, 0.06% by mass of monopotassium dihydrogen phosphate) was sterilized; 120 mL broth of the first-stage culturing was inoculated into the medium; and the culturing was carried out at 27° C. under supply of air (aeration rate: 1 vvm) for 1.5 days. The pH (25° C.) of the medium was maintained at 6.0 through addition of an appropriate amount of 3N sodium hydroxide solution.

### Recovery of Pellet-Form Filamentous Fungus

The pellet-form filamentous fungus culture mixture obtained in each of the aforementioned stages was filtered with gauze for one minute until filtrate dripping was stopped, to thereby produce a wet pellet-form filamentous fungus. The pellet-form filamentous fungus obtained in the second stage was immediately subjected to evaluation of fermentability.

### Culture Example 2

### Immobilization of Filamentous Fungus on Carrier

An immobilized filamentous fungus was prepared through the following two-stage culturing.

In the first stage, a 100-mL Erlenmeyer flask containing 30 mL of immobilization medium (5% by mass of glucose (product of Wako Pure Chemical Industries, Ltd.), 0.025% by mass of magnesium sulfate heptahydrate, 0.009% by mass of zinc sulfate heptahydrate, 0.2% by mass urea, 0.06% by mass of monopotassium dihydrogen phosphate) and five polyurethane foams (APG, product of Nisshinbo, size: 0.8 mm×0.8 mm) was sterilized; a spore suspension prepared in the same manner as in the case of the aforementioned pellet-form filamentous fungus was inoculated into the medium (2×10⁴ spores/mL); and the culturing was carried out at 35° C. and 200 r/m (PRXYg-98R, product of PRECI) for one day.

In the second stage, a 500-mL Erlenmeyer flask containing 100 mL of cell proliferation medium (10% by mass of glucose (product of Wako Pure Chemical Industries, Ltd.), 0.025% by mass of magnesium sulfate heptahydrate, 0.009% by mass of zinc sulfate heptahydrate, 0.1% by mass of urea, 0.06% by mass of monopotassium dihydrogen phosphate, 5% by mass of calcium carbonate) was sterilized; the filamentous fungus immobilized on the carrier in the first stage was inoculated into the medium; and culturing was carried out at 35° C. and 200 r/m (PRXYg-98R, product of PRECI) for two days.

### Recovery of Immobilized Filamentous Fungus

The immobilized filamentous fungus culture mixture obtained in each of the aforementioned stages was filtered with gauze for one minute until filtrate dripping was stopped, to thereby produce a wet immobilized filamentous fungus. The immobilized filamentous fungus obtained in the second stage was immediately subjected to evaluation of fermentability.

### Fermentation Example 1

### Evaluation of Fermentability

### Culture Method 1

Two L of lactic acid fermentation culture medium was added to a sterilized 2-L airlift-type fermentation vessel, and then the entire amount of the (wet) pellet-form filamentous fungus prepared in Culture Example 1 was added to the vessel. Immediately thereafter (i.e., O-hour culturing), sampling was carried out, and then culturing was carried out at 35° C. under supply of air (aeration rate: 1 vvm) for 14 days while sampling was performed periodically. The pH (25° C.) of the culture medium was maintained at 6.0 through addition of an appropriate amount of 3N sodium hydroxide solution. During fermentation, the lactic acid fermentation culture medium was continuously supplied into the fermentation vessel at a rate of 2 L/day while the same amount of the resultant fermentation mixture was removed from the fermentation vessel. Supply of the culture medium was carried out while the liquid level of the fermentation mixture was maintained constant by controlling a liquid recovery pump by means of a liquid level sensor. After completion of culturing, only the resultant fermentation mixture was recovered while the pellet-form filamentous fungus was left in the fermentation vessel by means of a sintered filter provided in the vessel.

### Fermentation Example 2

### Evaluation of Fermentability

### Culture Method 2

One-hundred mL of lactic acid fermentation culture medium was added to a sterilized 500-mL Erlenmeyer flask, and then the entire amount of the (wet) immobilized filamentous fungus prepared in Culture Example 2 was added to the flask. Immediately thereafter (i.e., O-hour culturing), sampling was carried out, and then culturing was carried out at 35° C. and 200 r/m (PRXYg-98R, product of PRECI) for two days. Sampling was carried out at the time when fermentation was completed, and then the immobilized filamentous fungus was recovered. The thus-recovered immobilized filamentous fungus was added to a sterilized 500-mL Erlenmeyer flask containing 100 mL of lactic acid fermentation culture medium. Subsequently, culturing was carried out at 35° C. and 200 r/m (PRXYg-98R, product of PRECI) for two days, and then the immobilized filamentous fungus was recovered. Thereafter, a batch culturing process was repeatedly carried out in the same manner as described above by use of the thus-recovered immobilized filamentous fungus.

### Evaluation Method

On the basis of analysis data of the fermentation mixture, the following three items were evaluated: (1) percent conversion of saccharide to lactic acid (P [%]), (2) percent conversion of saccharide to ethanol (Q [%]), and (3) percent conversion of saccharide to carbon dioxide generated through the aforementioned ethanol conversion (R [%]). These items were calculated as shown in Table 1 and the below-described formulas (1) to (3). In these formulas, G₀ represents the glucose concentration of a saccharide solution supplied. As shown in formula (4), S [-] represents the percent dilution of the fermentation mixture with a neutralizing agent. In the case of fermentation in the flask, the percent dilution S is 1, since the fermentation mixture is neutralized with calcium carbonate preliminarily incorporated into the culture medium.

TABLE 1

| | | Fermentation mixture |
|---|---|---|
| Concentration of | Glucose (Glc) | G |
| component | Lactic acid (LA) | L |
| [g/L] | Ethanol (EtOH) | E |

Percent conversion of saccharide to lactic acid

P [%]=L/(G₀×S−G)×100 (1)

Percent conversion of saccharide to ethanol

Q [%]=E/(G₀×S−G)×100 (2)

Percent conversion of saccharide to carbon dioxide generated through the aforementioned ethanol conversion

R [%]=Q×(CO₂ molecular weight/EtOH molecular weight)×100 (3)

Percent dilution of fermentation mixture with neutralizing agent

S [-]=1/(1+(L/90/3)) (4)

Percent maintenance of lactic acid production rate

T [%]=lactic acid production rate in sample [g/L/h]/management value of lactic acid production rate [g/L/h]×100

### Effect of Reducing Phosphate Concentration During Production of Lactic Acid

### Example 1

### Preparation of Pellet-Form Filamentous Fungus

A pellet-form filamentous fungus was prepared from filamentous fungus R. oryzae NBRC5384 in the manner as described above in Culture Example 1.

### Evaluation of Fermentability

Fermentability was evaluated in the manner as described in Fermentation Example 1 by use of a lactic acid fermentation culture medium containing glucose, urea, magnesium sulfate heptahydrate, and zinc sulfate heptahydrate at concentrations shown in Table 2. Glucose (product of Wako Pure Chemical Industries, Ltd.) was employed as a glucose source. The evaluation results are shown in Table 4.

### Example 2

The procedure of Example 1 was repeated, except that 0.5% by mass of sorbitan monolaurate (trade name: Rheodol SP-L10, product of Kao Corporation) was added to the PDB medium in the first-stage culturing, to thereby prepare a pellet-form filamentous fungus. The procedure of Example 1 was repeated, except that a lactic acid fermentation culture medium containing monopotassium dihydrogen phosphate, the phosphate ion concentration of which is set at 0.0014% by mass (0.15 mM), as shown in Table 2 was employed, to thereby evaluate fermentability. The evaluation results are shown in Table 4.

### Example 3

The procedure of Example 2 was repeated, except that a lactic acid fermentation culture medium containing monopotassium dihydrogen phosphate, the phosphate ion concentration of which is set at 0.0035% by mass (0.37 mM)), as shown in Table 2 was employed, to thereby prepare a pellet-form filamentous fungus, and to evaluate fermentability. The evaluation results are shown in Table 4.

### Comparative Example 1

The procedure of Example 1 was repeated, except that a lactic acid fermentation culture medium containing monopotassium dihydrogen phosphate, the phosphate ion concentration of which is set at 0.0070% by mass (0.73 mM), as shown in Table 3 was employed, to thereby prepare a pellet-form filamentous fungus, and to evaluate fermentability. The evaluation results are shown in Table 5.

### Comparative Example 2

The procedure of Example 1 was repeated, except that a lactic acid fermentation culture medium containing monopotassium dihydrogen phosphate, the phosphate ion concentration of which is set at 0.042% by mass (4.4 mM), as shown in Table 3 was employed, to thereby prepare a pellet-form filamentous fungus, and to evaluate fermentability. The evaluation results are shown in Table 5.

TABLE 2

| | | Example 1 | Example 2 | Example 3 |
|---|---|---|---|---|
| Concentration of | Glucose | 10% by mass | 10% by mass | 10% by mass |
| component in | KH₂PO₄ | None | 0.002% by mass | 0.005% by mass |
| culture medium | (as reduced to | | (0.0014% by mass) | (0.0035% by mass) |
| | phosphate ion) | | (0.15 mM) | (0.37 mM) |
| | Urea | 0.10 s % by mass | 0.10% by mass | 0.10% by mass |
| | | (17 mM) | (17 mM) | (17 mM) |
| | MgSO₄•7H₂O | 0.025% by mass | 0.025% by mass | 0.025% by mass |
| | ZnSO₄•7H₂O | 0.0090% by mass | 0.0090% by mass | 0.0090% by mass |

| | | | |
|---|---|---|---|
| Fermentation mode | 14 consecutive days | 14 consecutive days | 9 consecutive days |

TABLE 3

| | | Comparative | Comparative |
|---|---|---|---|
| | | Example 1 | Example 2 |
| Concentra- | Glucose | 10% by mass | 10% by mass |
| tion of | KH₂PO₄ | 0.010% by mass | 0.060% by mass |
| component | (as reduced to | (0.0070% by mass) | (0.042% by mass) |
| in culture | phosphate ion) | (0.73 mM) | (4.4 mM) |
| medium | Urea | 0.10% by mass | 0.10% by mass |
| | | (17 mM) | (17 mM) |
| | MgSO₄•7H₂O | 0.025% by mass | 0.025% by mass |
| | ZnSO₄•7H₂O | 0.0090% by mass | 0.0090% by mass |

| | | |
|---|---|---|
| Fermentation mode | 14 consecutive days | 14 consecutive days |

TABLE 4

| | | Example 1 | Example 2 | Example 3 |
|---|---|---|---|---|
| Evaluation | Lactic acid conversion | 87% | 85% | 84% |
| | Ethanol conversion | 0% | 0% | 0% |
| | CO₂ conversion | 0% | 0% | 0% |
| | Glucose consumption | 1.7 g/L/h | 0.8 g/L/h | 1.0 g/L/h |
| | rate | | | |
| | Lactic acid production | 1.5 g/L/h | 0.7 g/L/h | 0.8 g/L/h |
| | rate | | | |

TABLE 5

| | | Comparative | Comparative |
|---|---|---|---|
| | | Example 1 | Example 2 |
| Evaluation | Lactic acid conversion | 66% | 69% |
| | Ethanol conversion | 6.5% | 10% |
| | CO₂ conversion | 6.2% | 9.9% |
| | Glucose consumption | 1.8 g/L/h | 3.1 g/L/h |
| | rate | | |
| | Lactic acid production | 1.2 g/L/h | 2.2 g/L/h |
| | rate | | |

As is clear from Tables 4 and 5, when the phosphate ion concentration of the culture medium falls within the range specified in the present invention, no ethanol production is observed, whereas when the phosphate ion concentration of the culture medium falls outside the range specified in the present invention, ethanol is produced, and lactic acid conversion with respect to consumed glucose is considerably reduced.

### Example 4

### Preparation of Immobilized Filamentous Fungus

An immobilized filamentous fungus was prepared from filamentous fungus R. oryzae NBRC5384 in the manner as described above in Culture Example 2.

### Evaluation of Fermentability

Fermentability was evaluated for 50 days (25 cycles) in the manner as described in Fermentation Example 2 by use of a lactic acid fermentation culture medium containing glucose, urea, magnesium sulfate heptahydrate, and zinc sulfate heptahydrate at concentrations shown in Example 1 (Table 2). Glucose (product of Wako Pure Chemical Industries, Ltd.) was employed as a glucose source. The evaluation results are shown in Table 6.

### Example 5

### Preparation of Immobilized Filamentous Fungus

An immobilized filamentous fungus was prepared from filamentous fungus R. oryzae NBRC5384 in the manner as described above in Culture Example 2.

### Evaluation of Fermentability

Fermentability was evaluated for 36 days (18 cycles) in the manner as described in Fermentation Example 2 by use of a lactic acid fermentation culture medium containing glucose, urea, magnesium sulfate heptahydrate, and zinc sulfate heptahydrate at concentrations shown in Example 1 (Table 2). Thereafter, the immobilized filamentous fungus was recovered. The management value of lactic acid production rate was set at 1.6 [g/L/h], and, 36 days later, the lactic acid production rate was 1.3 [g/L/h]. Thereafter, the culture medium was replaced with a lactic acid fermentation culture medium shown in Comparative Example 2 (Table 3), and culturing was carried out for two days by use of the recovered immobilized filamentous fungus, followed by recovery of the immobilized filamentous fungus. Subsequently, fermentability was evaluated for 12 days (6 cycles) by employing the recovered immobilized filamentous fungus, and replacing the culture medium with a lactic acid fermentation culture medium shown in Example 1 (Table 2). Glucose (product of Wako Pure Chemical Industries, Ltd.) was employed as a glucose source. The evaluation results are shown in Table 6.

TABLE 6

| | | Example 4 | Example 5 |
|---|---|---|---|
| Evaluation | Lactic acid conversion | 81% | 82% |
| | Ethanol conversion | 12.9% | 10.4% |
| | CO₂ conversion | 12.3% | 10.0% |
| 36 days later | Percent maintenance of lactic acid | 87.7% | 82.9% |
| | production rate | | |
| 50 days later | Glucose consumption rate | 1.0 g/L/h | 2.0 g/L/h |
| 50 days later | Lactic acid production rate | 0.8 g/L/h | 1.7 g/L/h |
| 50 days later | Percent maintenance of lactic acid | 49.9% | 104.5% |
| | production rate | | |

As is clear from Table 6, even when the immobilized filamentous fungus is employed, similar to the case of the pellet-form filamentous fungus, lactic acid production can be continued while lactic acid conversion is maintained at high level. As is also clear from Table 6, in the case where fermentation is temporarily carried out in a culture medium having a high phosphate ion concentration when lactic acid production rate is lowered to 50 to 95% in the first fermentation step, cells are activated, and when the third fermentation step, which is similar to the first fermentation step, is carried out by use of the thus-activated cells, lactic acid production can be continued over a longer period of time while lactic acid conversion and lactic acid production rate are maintained at high levels.

1. A method for producing lactic acid, the method comprising a first fermentation step of carrying out fermentation by use of one or more fungus cells selected from the group consisting of pellet-form filamentous fungus cells and immobilized filamentous fungus cells in a liquid culture medium having a phosphate ion concentration of less than 0.007% by mass and containing a carbon source, to thereby produce lactic acid.

2. The method according to claim 1, wherein the phosphate ion concentration of the liquid culture medium employed in the first fermentation step is from 0 to 0.006% by mass.

3. The method according to claim 1, wherein the phosphate ion concentration of the liquid culture medium employed in the first fermentation step is from 0 to 0.005% by mass.

4. The method for producing lactic acid according to claim 1, which comprises a second fermentation step of carrying out fermentation by use of the cells employed in the first fermentation step in a liquid culture medium having a phosphate ion concentration of from 0.007% by mass to 1% by mass and containing a carbon source, the second fermentation step being carried out at the time when the percent maintenance of lactic acid production rate becomes from 50 to 95% in the first fermentation step.

5. The method for producing lactic acid according to claim 4, wherein the percent maintenance of lactic acid production rate is from 60 to 90%.

6. The method for producing lactic acid according to claim 4, wherein the percent maintenance of lactic acid production rate is determined by use of the following formula (i):
T [%]=Vt [g/L/h]/Vi [g/L/h]×100 (i)
[wherein T represents the percent maintenance of lactic acid production rate [%]; Vt represents lactic acid production rate in a sample [g/L/h]; and Vi represents the management value of lactic acid production rate [g/L/h]].

7. The method for producing lactic acid according to claim 6, wherein the management value of lactic acid production rate is from 0.3 to 30 g/L/h.

8. The method for producing lactic acid according to claim 4, wherein the phosphate ion concentration of the liquid culture medium employed in the second fermentation step is from 0.01 to 0.09% by mass.

9. The method for producing lactic acid according to claim 4, wherein the second fermentation step is carried out for from 12 to 120 hours.

10. The method for producing lactic acid according to claim 4, which comprises a third fermentation step of carrying out fermentation by use of the cells employed in the second fermentation step in a liquid culture medium having a phosphate ion concentration of less than 0.007% by mass and containing a carbon source, the third fermentation step being carried out after completion of the second fermentation step.

11. The method for producing lactic acid according to claim 10, wherein the phosphate ion concentration of the liquid culture medium employed in the third fermentation step is from 0 to 0.005% by mass.

12. The method for producing lactic acid according to claim 1, which comprises a step of preparing one or more fungus cells selected from the group consisting of pellet-form filamentous fungus cells and immobilized filamentous fungus cells, the step being carried out before the first fermentation step.

13. The method for producing lactic acid according to claim 1, wherein the filamentous fungus belongs to the genus Rhizopus.

14. The method for producing lactic acid according to claim 1, wherein the filamentous fungus is one or more species selected from the group consisting of Rhizopus oryzae, spergillus oryzae, Aspergillus niger, Aspergillus terreus, and Mucor mandshuricus.

15. The method for producing lactic acid according to claim 14, wherein the filamentous fungus is Rhizopus oryzae.

16. The method for producing lactic acid according to claim 1, wherein the carbon source is a saccharide.

17. The method for producing lactic acid according to claim 16, wherein the saccharide is one or more species selected from the group consisting of glucose, fructose, xylose, and sucrose.

18. The method for producing lactic acid according to claim 16, wherein the saccharide is one or more species selected from the group consisting of glucose and fructose.

19. The method for producing lactic acid according to claim 16, wherein the saccharide is one or more species selected from the group consisting of a sugar solution derived from starch, syrup, and a sugar solution derived from lignocellulosic biomass.

20. The method for producing lactic acid according to claim 10, wherein one or more steps of the first, second, and third fermentation steps are carried out through a continuous process in which a specific amount of a liquid culture medium is supplied to a fermentation vessel at a specific rate while the same amount of the resultant fermentation mixture is removed from the fermentation vessel.
